(19) United States
(12) Patent Application Publication
(10) Pub. No.: US 2021/0353360 A1
(43) Pub. Date: Nov. 18, 2021

(54) CRANIOTOMY SIMULATION DEVICE, METHOD, AND PROGRAM

(71) Applicant: FUJIFILM Corporation, Tokyo (JP)

(72) Inventor: Hirotaka ITO, Tokyo (JP)

(73) Assignee: FUJIFILM Corporation, Tokyo (JP)

(21) Appl. No.: 17/384,806

(22) Filed: Jul. 26, 2021

Related U.S. Application Data
(63) Continuation of application No. PCT/JP2019/036904, filed on Sep. 20, 2019.

(30) Foreign Application Priority Data
Feb. 8, 2019 (JP) 2019-022083

(51) Int. Cl. A61B 34/10; G06T 19/20; G06T 7/00; G06T 7/30

(52) U.S. Cl. CPC A61B 34/10; G06T 19/20; G06T 7/0012; G06T 7/30; G06T 2207/30016; A61B 2034/107; G06T 2219/2012; G06T 2210/41; G06T 2219/2016; A61B 2034/105

(57) ABSTRACT

In a craniotomy simulation device, method, and program, a path to an abnormal area can be efficiently determined for a simulation of a craniotomy. A path derivation unit derives, in a three-dimensional image of a brain of a subject including an abnormal area, at least one path from the abnormal area to a surface of the brain through a cerebral sulcus in the brain. A craniotomy pattern setting unit sets a craniotomy pattern for tracing the path, on a surface of a head of the subject included in the three-dimensional image.

### CROSS-REFERENCE TO RELATED APPLICATIONS

This application is a Continuation of PCT International Application No. PCT/JP2019/036904 filed on Sep. 20, 2019, which claims priority under 35 U.S.C § 119(a) to Japanese Patent Application No. 2019-022083 filed on Feb. 8, 2019. Each of the above application(s) is hereby expressly incorporated by reference, in its entirety, into the present application.

### BACKGROUND OF THE INVENTION

### 1. Field of the Invention

The present disclosure relates to a craniotomy simulation device, method, and non-transitory computer recording medium storing a program for performing a simulation of a craniotomy of a brain using a three-dimensional image of a head.

### 2. Description of the Related Art

In recent years, surgical simulations using three-dimensional medical images have been actively performed. In a surgical simulation, a tissue or an organ for which surgery is to be performed, and a peripheral structure thereof are visualized in a medical image, and a procedure to be performed in an actual surgery is simulated before the surgery. For example, in a surgery for excision of a tumor in the brain, the tumor is excised by craniotomy for opening the brain. To simulate the craniotomy, tissues, such as the skin, skull, brain, cerebral arteries, cerebral veins, cranial nerves, and tumor, are extracted from a three-dimensional image of a CT (Computed Tomography) image or an MM (Magnetic Resonance Imaging) image, and a three-dimensional image in which these tissues are visualized is generated. Then, the generated three-dimensional image is used to simulate skin incision, craniotomy, and a path from the position of craniotomy to the tumor by calculation or the like with a computer, and a surgical plan is created with reference to the simulation.

On the other hand, for a craniotomy, a pattern of skin incision in which important organs of the head, such as the eyes, noise, and mouth, are not incised is adopted in terms of aesthetic results after surgery. In general, the pattern of skin incision is determined such that a position hidden by the hair is incised. A portion of the brain has to be incised to reach an abnormal area, such as a tumor, from the incision position. However, incision of the brain may result in sequelae. For this reason, a path for reaching the abnormal area from the craniotomy position is simulated using cerebral sulci as much as possible.

For example, JP2017-514637A proposes a method for determining a cannulation trajectory for inserting a cannula into the brain along a cerebral sulcus specified in a three-dimensional image. JP2016-517288A proposes a method for performing a simulation of a target position of a cerebral sulcus and a surgical path for approaching the tissue on the basis of a three-dimensional image. JP2013-111422A proposes a method for identifying a cerebral sulcus that is not to be used from a three-dimensional image of a brain on the basis of position information of an epileptic focus and position information of a cerebral blood vessel and a cerebral sulcus.

### SUMMARY OF THE INVENTION

In the methods described in JP2017-514637A, JP2016-517288A, and JP2013-111422A above, a simulation for reaching an abnormal area from a determined incision position of the skin is performed. That is, a simulation is performed in which skin incision is made at the determined incision position of the skin, followed by bone incision, a cerebral sulcus is selected, and a tumor is reached through the cerebral sulcus. However, there may be a case where a cranial nerve and an important blood vessel are present on a path determined by a simulation. There may also be a case where the simulated path does not meet the desires of a doctor who performs surgery. In such cases, it is necessary to perform the simulation again by changing the incision position of the skin or the like. For this reason, the methods described in JP2017-514637A, JP2016-517288A, and JP2013-111422A make it difficult to efficiently determine a path to an abnormal area.

The present disclosure has been made in view of the circumstances described above, and it is an object thereof to enable efficient determination of a path to an abnormal area for a simulation of a craniotomy.

A craniotomy simulation device according to the present disclosure includes
a path derivation unit that derives, in a three-dimensional image of a brain of a subject including an abnormal area, at least one path from the abnormal area to a surface of the brain through a cerebral sulcus in the brain, and
a craniotomy pattern setting unit that sets a craniotomy pattern for tracing the path, on a surface of a head of the subject included in the three-dimensional image.

In the craniotomy simulation device according to the present disclosure, the craniotomy pattern setting unit may set the craniotomy pattern on the basis of a template selected from respective templates representing a plurality of standard craniotomy patterns in accordance with a position of the path on the surface of the brain.

The “template representing a craniotomy pattern” is obtained by superimposing a position and shape of standard skin incision and a position and shape of bone incision, which are used in a craniotomy, on a standard head model.

In the craniotomy simulation device according to the present disclosure, furthermore, the craniotomy pattern setting unit may correct the selected template in accordance with a shape of the head of the subject to set the craniotomy pattern.

In the craniotomy simulation device according to the present disclosure, furthermore, the path derivation unit may select at least one cerebral sulcus within a predetermined range from a position of the abnormal area and derive the path that passes through the selected cerebral sulcus.

In the craniotomy simulation device according to the present disclosure, furthermore, the path derivation unit may derive the path that passes through a cerebral sulcus other than a cerebral sulcus selected in advance.

In the craniotomy simulation device according to the present disclosure, furthermore, the path derivation unit may derive the path that avoids an organ designated in advance.

The craniotomy simulation device according to the present disclosure may further include a display control unit that displays a three-dimensional image of the head of the subject for which the craniotomy pattern is set, on a display unit as a simulation image.

In the craniotomy simulation device according to the present disclosure, furthermore, the display control unit may display, on the display unit, the simulation image in which a point of view is shifted from the surface of the head of the subject to the abnormal area along the path.

In the craniotomy simulation device according to the present disclosure, furthermore, the display control unit may display, on the display unit, the simulation image in which the path is highlighted.

In the craniotomy simulation device according to the present disclosure, furthermore, the path derivation unit may derive a plurality of the paths,
the craniotomy pattern setting unit may set the craniotomy pattern for each of the plurality of paths, and
the display control unit may sort the plurality of paths in accordance with a distance from the abnormal area to a cerebral sulcus and display a sorting result on the display unit.

In the craniotomy simulation device according to the present disclosure, furthermore, the path derivation unit may derive a plurality of the paths,
the craniotomy pattern setting unit may set the craniotomy pattern for each of the plurality of paths, and
the display control unit may sort the plurality of paths in accordance with a distance from the abnormal area to the surface of the brain and display a sorting result on the display unit.

A craniotomy simulation method according to the present disclosure includes
deriving, in a three-dimensional image of a brain of a subject including an abnormal area, at least one path from the abnormal area to a surface of the brain through a cerebral sulcus in the brain, and
setting a craniotomy pattern for tracing the path, on a surface of a head of the subject included in the three-dimensional image.

There may be provided a non-transitory computer recording medium storing a program for causing a computer to execute the craniotomy simulation method according to the present disclosure.

Another craniotomy simulation device according to the present disclosure includes
a memory that stores instructions to be executed by the computer, and
a processor configured to execute the stored instructions,
wherein the processor
derives, in a three-dimensional image of a brain of a subject including an abnormal area, at least one path from the abnormal area to a surface of the brain through a cerebral sulcus in the brain, and
sets a craniotomy pattern for tracing the path, on a surface of a head of the subject included in the three-dimensional image.

According to the present disclosure, a path to an abnormal area can be efficiently determined for a simulation of a craniotomy.

### BRIEF DESCRIPTION OF THE DRAWINGS

FIG. 1 is a hardware configuration diagram illustrating an overview of a diagnosis support system to which a craniotomy simulation device according to an embodiment of the present disclosure is applied;

FIG. 2 is a schematic block diagram illustrating a configuration of the craniotomy simulation device according to this embodiment;

FIG. 3 is a diagram illustrating an example three-dimensional image of a head;

FIG. 4 is a diagram illustrating a brain included in the three-dimensional image using the three-dimensional coordinates;

FIG. 5 is a diagram illustrating a tomographic image of the brain for explaining cerebral sulci;

FIG. 6 is a diagram illustrating a tomographic image of the brain for explaining selection of a cerebral sulcus;

FIG. 7 is a diagram for explaining the derivation of a path;

FIG. 8 is a diagram illustrating example templates;

FIG. 9 is a diagram illustrating the position of a derived path on the surface of the brain;

FIG. 10 is a diagram illustrating a state in which a template subjected to alignment is overlaid on the head of the subject;

FIG. 11 is a diagram illustrating a simulation image, which is a displayed three-dimensional image of the head of the subject;

FIG. 12 is a diagram illustrating a simulation image;

FIG. 13 is a diagram illustrating a simulation image;

FIG. 14 is a diagram illustrating a simulation image;

FIG. 15 is a diagram illustrating a simulation image;

FIG. 16 is a diagram illustrating a simulation image;

FIG. 17 is a flowchart illustrating a process performed in this embodiment;

FIG. 18 is a diagram illustrating a tomographic image of the brain for explaining selection of a cerebral sulcus;

FIG. 19 is a diagram illustrating the position of a derived path on the surface of the brain; and

FIG. 20 is a diagram illustrating a simulation image including a sorting result.

### DESCRIPTION OF THE PREFERRED EMBODIMENTS

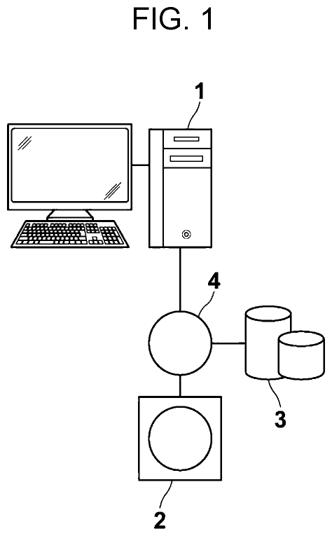

The following describes an embodiment of the present disclosure with reference to the drawings. FIG. 1 is a hardware configuration diagram illustrating an overview of a diagnosis support system to which a craniotomy simulation device according to an embodiment of the present disclosure is applied. As illustrated in FIG. 1, in the diagnosis support system, a craniotomy simulation device 1 according to this embodiment, a three-dimensional imaging device 2, and an image storage server 3 are communicably connected to each other via a network 4.

The three-dimensional imaging device 2 is a device that captures an image of an area of the subject to be diagnosed to generate a three-dimensional image representing the area, and specific examples of the device include a CT device, an MRI device, and a PET (Positron Emission Tomography) device. The three-dimensional image generated by the three-dimensional imaging device 2 is transmitted to and saved in the image storage server 3. In this embodiment, it is assumed that the diagnostic target area of a patient, which is the subject, is a brain, the three-dimensional imaging device 2 is an MM device, and the three-dimensional imaging device 2 generates an MM image of the head of the patient, which is the subject, as a three-dimensional image.

The image storage server 3 is a computer that saves and manages various data, and includes a large-capacity external storage device and database management software. The image storage server 3 communicates with another device via the network 4, which is wired or wireless, and transmits and receives image data and the like. Specifically, the image storage server 3 acquires various data, including image data of the three-dimensional image or the like generated by the three-dimensional imaging device 2, via a network, saves the data in a recording medium such as a large-capacity external storage device, and manages the data. The storage format of image data and communication between devices via the network 4 are based on a protocol such as DICOM (Digital Imaging and Communication in Medicine).

In a three-dimensional image G0 saved in the image storage server 3, the positions of abnormal areas such as tumors and aneurysms included in the brain are assumed to have been identified by an abnormal area detection device (not illustrated). The abnormal areas may be identified by CAD (Computer-Aided Diagnosis) using a discriminator that has performed learning using deep learning or the like, but this is not limiting. The doctor may read the displayed three-dimensional image G0 to identify the abnormal areas. Information on the identified abnormal areas is saved in the image storage server 3 together with the three-dimensional image G0.

The craniotomy simulation device 1 is implemented by one computer into which a craniotomy simulation program of the present disclosure is installed. The computer may be a workstation or a personal computer to be directly operated by a doctor who performs diagnosis, or may be a server computer connected to such a device via a network. The craniotomy simulation program is recorded on and distributed through a recording medium such as a DVD (Digital Versatile Disc) or a CD-ROM (Compact Disk Read Only Memory), and is installed into the computer from the recording medium. Alternatively, the craniotomy simulation program is stored in a storage device of a server computer connected to a network or in a network storage in an externally accessible state, and is downloaded and installed into a computer used by the doctor in response to a request.

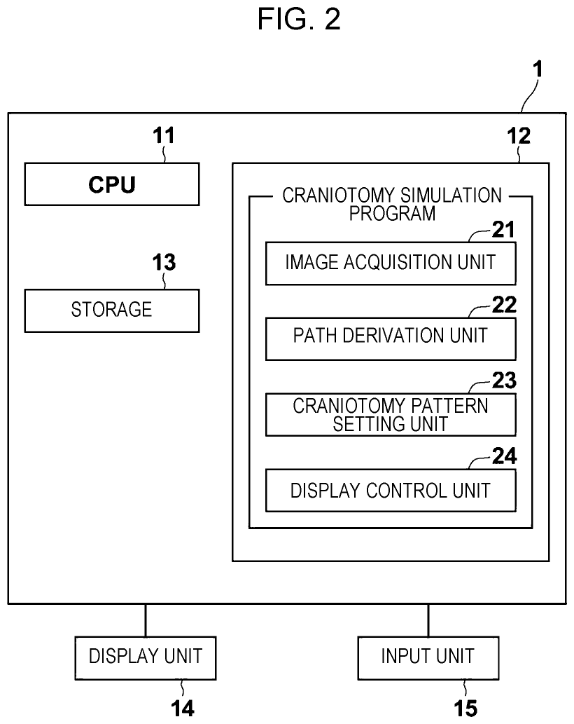

FIG. 2 is a diagram illustrating a schematic configuration of a craniotomy simulation device according to an embodiment of the present disclosure, which is implemented by installing the craniotomy simulation program into a computer. As illustrated in FIG. 2, the craniotomy simulation device 1 includes, as a configuration of a standard workstation, a CPU (Central Processing Unit) 11, a memory 12, and a storage 13. The craniotomy simulation device 1 is connected to a display unit 14 and an input unit 15 such as a mouse and a keyboard.

The storage 13 stores the three-dimensional image G0 of the subject, which is acquired from the image storage server 3 via the network 4, and various types of information including information necessary for processing. The storage 13 is constituted by, for example, an HDD (Hard Disc Drive) or an SSD (Solid State Drive). In this embodiment, it is assumed that the three-dimensional image G0 in which the head of the subject is a target area is stored in the storage 13.

In this aspect, the memory 12 stores the craniotomy simulation program. In this case, the memory 12 may be constituted by a non-volatile memory. The craniotomy simulation program specifies, as processes to be executed by the CPU 11, an image acquisition process for acquiring the three-dimensional image G0 including an abnormal area, a path derivation process for deriving at least one path from the abnormal area to a surface of the brain through a cerebral sulcus in the brain in the three-dimensional image G0, a craniotomy pattern setting process for setting a craniotomy pattern for tracing the path, on the surface of the head of the patient included in the three-dimensional image G0, and a display control process for displaying, on the display unit 14, the three-dimensional image G0 of the head of the patient on which the craniotomy pattern is superimposed. In another aspect, the craniotomy simulation program saved in the storage 13 may be invoked by the CPU 11, temporarily stored in the memory 12, and then executed. In this case, the memory 12 is constituted by a RAM (Random Access Memory).

The CPU 11 executes these processes in accordance with the program, and thus the computer functions as an image acquisition unit 21, a path derivation unit 22, a craniotomy pattern setting unit 23, and a display control unit 24.

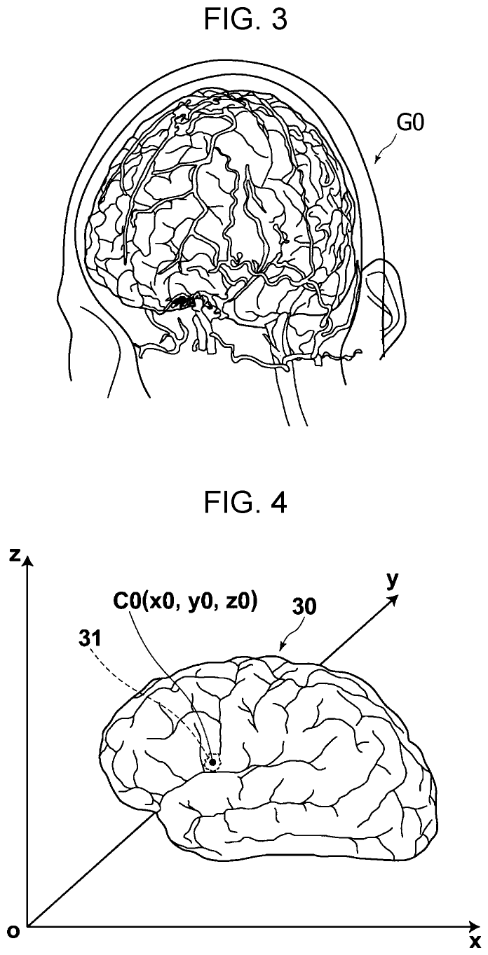

The image acquisition unit 21 acquires the three-dimensional image G0 of the head of the patient, which is the subject, from the image storage server 3. If the three-dimensional image G0 has already been stored in the storage 13, the image acquisition unit 21 may acquire the three-dimensional image G0 from the storage 13. FIG. 3 is a diagram illustrating an example of the three-dimensional image G0 of the head. FIG. 3 illustrates a state in which the three-dimensional image G0 is displayed using volume rendering such that, among organs such as the skin, muscles, skull, brain, nerves, cerebral arteries, and cerebral veins, only portions of the skin, muscles, and skull are shown as transparent in the three-dimensional image G0. The three-dimensional image G0 acquired by the image acquisition unit 21 includes an abnormal area. Thus, the image acquisition unit 21 also acquires information on the abnormal area together with the three-dimensional image G0. The information on the abnormal area includes the coordinates of the center-of-gravity position of the abnormal area in the three-dimensional coordinates representing the three-dimensional image G0 and the coordinates of pixels identified as the abnormal area.

FIG. 4 is a diagram illustrating the brain included in the three-dimensional image G0 using the three-dimensional coordinates. As illustrated in FIG. 4, the position of each pixel (voxel) in a brain 30 can be represented by three-dimensional coordinates with respect to an origin O in the three-dimensional image G0. The brain 30 includes a tumor 31 as an abnormal area. FIG. 4 illustrates a center-of-gravity position C0 (coordinates (x0, y0, z0)) of the tumor 31.

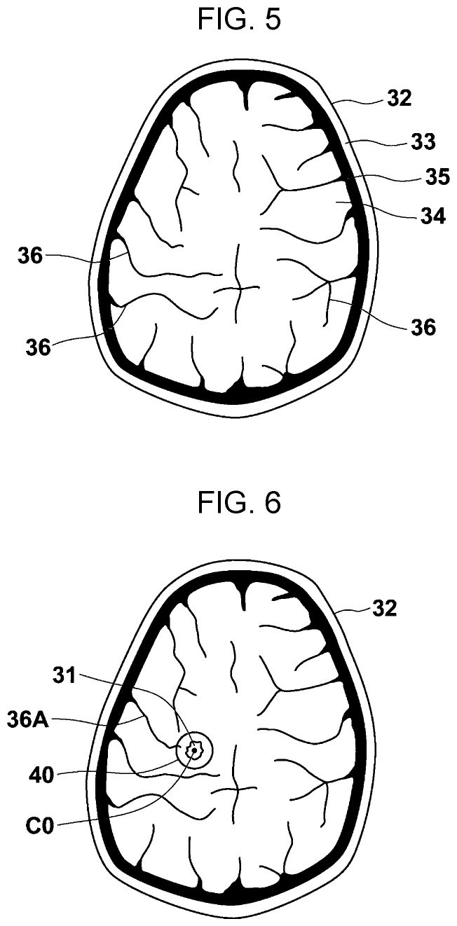

For a craniotomy of a brain, a portion of the brain has to be incised to reach the abnormal area from the incision position of the skin. However, incision of the brain may result in sequelae. For this reason, it is necessary to reach the abnormal area using cerebral sulci as much as possible. FIG. 5 is a diagram illustrating a tomographic image of the brain for explaining cerebral sulci. FIG. 5 illustrates a tomographic image 32 of a cross section viewed from the feet of the subject. As illustrated in FIG. 5, the tomographic image 32 of the brain includes a skull 33 and brain parenchyma 34. The space between the skull 33 and the brain parenchyma 34 is filled with a cerebrospinal fluid 35. The brain parenchyma 34 includes a plurality of cerebral sulci 36. Due to the cerebrospinal fluid 35 inside the cerebral sulci 36, the brain parenchyma 34 and the cerebral sulci 36 have different signal values in the three-dimensional image G0. In the three-dimensional image G0, accordingly, the positions of the cerebral sulci 36 in the brain parenchyma 34 can be identified.

As illustrated in FIG. 6, the tomographic image 32 of the brain is assumed to include the tumor 31 existing in the brain. FIG. 6 also illustrate a tomographic image of a cross section viewed from the feet of the subject. The path derivation unit 22 selects at least one cerebral sulcus within a predetermined range from the center-of-gravity position C0 of the tumor 31. In this embodiment, the path derivation unit 22 sets a sphere 40 having a predetermined radius centered on the center-of-gravity position C0 of the tumor 31, and selects at least one cerebral sulcus in the sphere 40. The sphere 40 is a circle in a tomographic image. In FIG. 6, there is only a cerebral sulcus 36A in the sphere 40. Thus, the path derivation unit 22 selects the cerebral sulcus 36A.

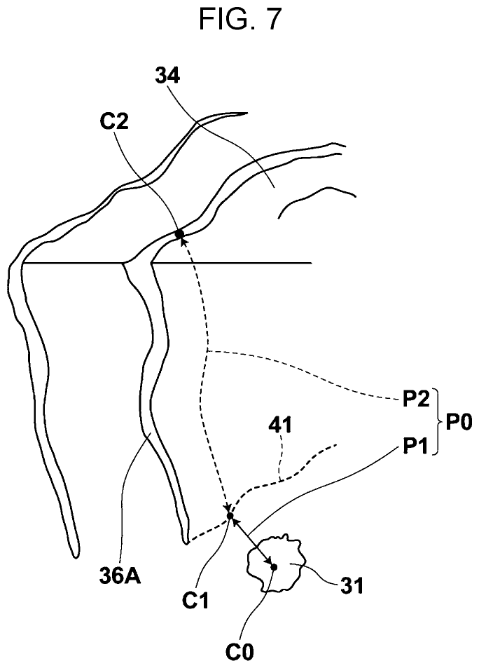

Then, the path derivation unit 22 derives a shortest distance P1 from the center-of-gravity position C0 of the tumor 31 to the selected cerebral sulcus 36A to derive a path. FIG. 7 is a cross-sectional view of the cerebral sulcus 36A for explaining the derivation of a path. In FIG. 7, the width of the cerebral sulcus 36A is illustrated to be larger than the actual one for convenience of explanation. As illustrated in FIG. 7, the brain parenchyma 34 is not completely divided by the cerebral sulci 36, but is continuous at the back of the cerebral sulci 36. Thus, the selected cerebral sulcus 36A has a bottom 41. The path derivation unit 22 derives distances between the coordinate values of individual pixel positions in the bottom 41 of the cerebral sulcus 36A and the coordinate values of the center-of-gravity position C0 of the tumor 31, and derives a distance that is the smallest among the distances as the shortest distance P1. Then, the path derivation unit 22 identifies a position C1 in the bottom 41 at which the shortest distance P1 is derived.

Further, the path derivation unit 22 derives a shortest distance P2 from the position C1 to the surface of the brain through the cerebral sulcus 36A. Specifically, the path derivation unit 22 derives distances between the coordinate values of individual positions of the cerebral sulcus 36A on the cerebral surface and the coordinate values of the position C1. The derived distances are distances passing through the cerebral sulcus 36A. The cerebral sulcus 36A is not the brain parenchyma, but is visible on the surface of the brain. Thus, a position of the cerebral sulcus 36A on the cerebral surface means a position of the cerebral sulcus 36A visible on the surface of the brain. The path derivation unit 22 derives the shortest distance P2 among the derived distances, and identifies the position of the cerebral sulcus 36A on the cerebral surface at which the shortest distance P2 is obtained as a start position C2. Accordingly, a path P0 (=P1+P2) from the tumor 31 to the start position C2 on the surface of the brain through the cerebral sulcus 36A is derived.

The craniotomy pattern setting unit 23 sets a craniotomy pattern for tracing the path P0, on the surface of the head of the patient included in the three-dimensional image G0. Thus, in this embodiment, respective templates representing a plurality of standard craniotomy patterns are stored in the storage 13. The craniotomy pattern setting unit 23 sets a craniotomy pattern from the templates stored in the storage 13, on the basis of a template selected in accordance with the start position C2 of the path P0 on the surface of the brain.

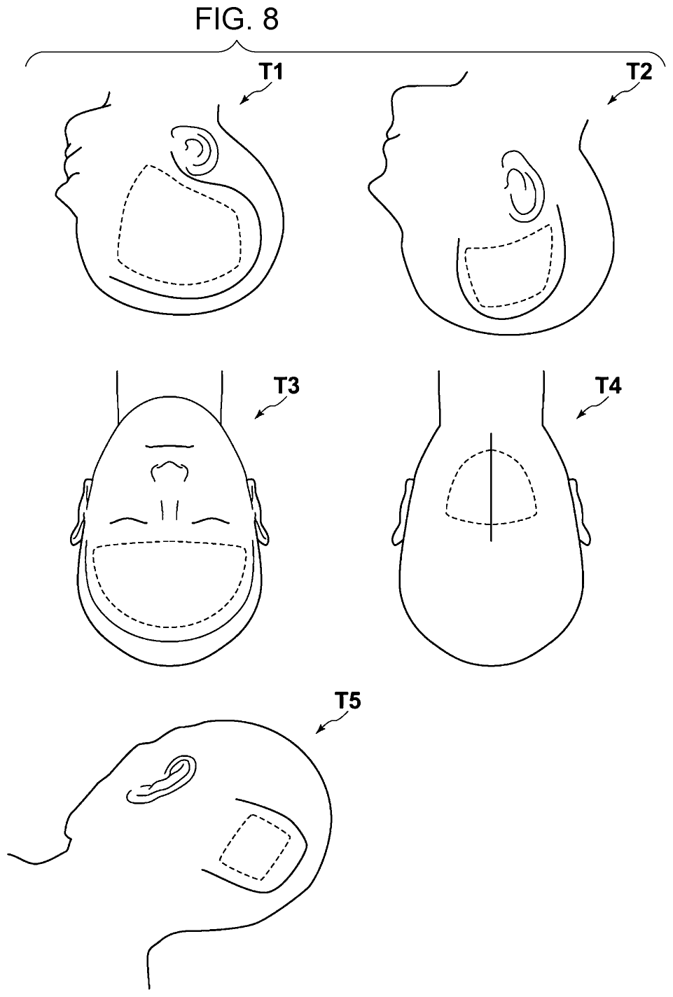

FIG. 8 is a diagram illustrating example templates. As illustrated in FIG. 8, templates T1 to T5 are each obtained by superimposing a position and shape of standard skin incision and a position and shape of bone incision, which are used in a craniotomy, on a standard head model. The templates T1 to T5 are three-dimensional images. In each of the templates T1 to T5 illustrated in FIG. 8, the incision line of the skin is indicated by a solid line, and the incision line of the skull is indicated by a broken line. While FIG. 8 illustrates five types of templates T1 to T5, the number of templates is not limited to this. Since each surgical operator has a preferred craniotomy pattern, it is also possible to store a template desired by each surgical operator in the storage 13. Although the templates T1, T2, and T5 are each a template for only one of the left and right sides of the head, templates for both sides of the head are actually prepared.

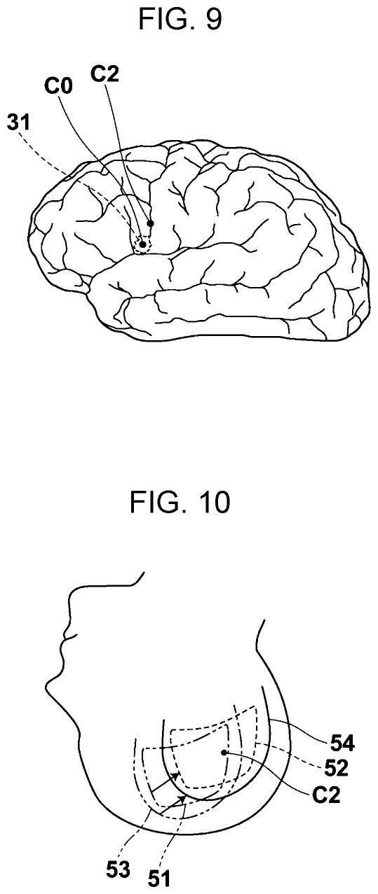

The craniotomy pattern setting unit 23 selects an appropriate template from the plurality of templates T1 to T5 in accordance with the start position C2 of the path P0 on the surface of the brain. In this embodiment, as illustrated in FIG. 9, if it is assumed that the start position C2 of the path P0 on the surface of the brain has been derived, the craniotomy pattern setting unit 23 selects a template indicating the craniotomy pattern for the position closest to the start position C2. In this embodiment, since the start position C2 is located in the right temporal region of the brain of the subject, the craniotomy pattern setting unit 23 selects the template T2 for the craniotomy of the right temporal region.

Further, the craniotomy pattern setting unit 23 corrects the selected template in accordance with the start position C2 and the shape of the head of the subject to set a craniotomy pattern. Specifically, the craniotomy pattern setting unit 23 displaces and deforms the incision lines of the skin and the skull, which are included in the selected template T2, in accordance with the start position C2 and the shape of the head of the subject to correct the template T2. At this time, the craniotomy pattern setting unit 23 aligns the position of the head included in the selected template T2 with the position of the head of the subject included in the three-dimensional image G0. At this time, any alignment method such as rigid alignment and non-rigid alignment can be used. FIG. 10 is a diagram illustrating a state in which a template subjected to alignment is overlaid on the head of the subject.

The craniotomy pattern setting unit 23 shifts the incision line 51 of the skull in the template T2, which is indicated by an imaginary line, to the position of an incision line 52, which is indicated by a broken line, so that the center of a region surrounded by the incision line 51 matches the start position C2 in the brain of the subject. Then, an incision line 53 of the skin in the template T2, which is indicated by an imaginary line, is shifted to the position of an incision line 54, which is indicated by a solid line, so as to be appropriate for the shifted incision line 52 of the skull. As a result, a craniotomy pattern is set in the image of the head of the subject.

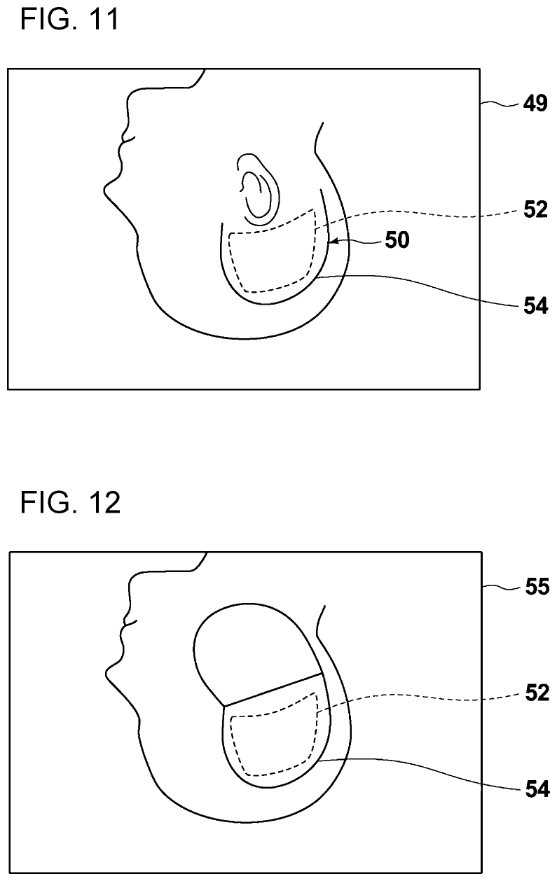

The display control unit 24 displays a simulation image, which is a three-dimensional image of the head of the subject for which the craniotomy pattern is set, on the display unit 14. The display control unit 24 appropriately sets transparency and a color template and displays the simulation image using volume rendering. FIG. 11 is a diagram illustrating a simulation image. In a simulation image 49 illustrated in FIG. 11, the skin is shown opaque, and a craniotomy pattern 50 for the skin is superimposed on the head. The craniotomy pattern 50 includes the incision line 52 of the skull and the incision line 54 of the skin.

In this embodiment, a simulation for performing a craniotomy and reaching the tumor 31 is performed in response to an instruction from the input unit 15. Accordingly, when the operator provides an instruction to start the simulation using the input unit 15, as illustrated in FIG. 12, the display control unit 24 displays, on the display unit 14, a simulation image 55 in which the skin is incised along the incision line 54 of the skin and is rolled up. FIG. 12 illustrates a state in which the incised skin is rolled up such that the skull is revealed. The incision line 52 of the skull is superimposed on the skull. Examples of the instruction from the input unit 15 include an instruction to rotate the wheel of the mouse, and an instruction to press an arrow key of the keyboard, but this is not limiting.

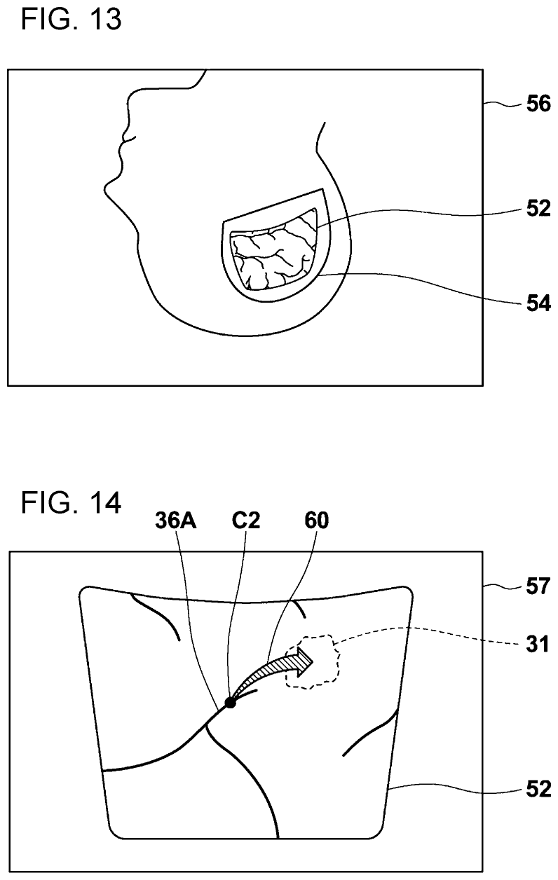

When the operator provides an instruction using the input unit 15, furthermore, as illustrated in FIG. 13, the display control unit 24 displays, on the display unit 14, a simulation image 56 in which the skull is incised along the incision line 52 and a portion of the skull is removed as a result of the incision. In FIG. 13, the incised skin is not illustrated. In the simulation image 56 illustrated in FIG. 13, the brain is revealed from the portion of the skull that is removed as a result of the incision.

The operator can provide an instruction using the input unit 15 to enlarge or reduce, rotate, and the like the image displayed on the display unit 14. FIG. 14 is a diagram illustrating a simulation image in which an incised region in FIG. 13 is enlarged. As illustrated in FIG. 14, in a simulation image 57, the region surrounded by the incision line 52 is enlarged, and the brain is revealed in the enlarged region. On the brain, the start position C2 is indicated by a black circle, and the tumor 31 is shown translucent. In FIG. 14, a translucent object is indicated by a broken line. A path 60 from the start position C2 to the tumor 31 through the cerebral sulcus 36A is indicated by an arrow.

The operator can also provide an instruction using the input unit 15 to shift the position of the point of view from the start position C2 toward the tumor 31 along the path 60. When this instruction is provided, the display control unit 24 gradually decreases the transparency of the three-dimensional image G0 to 0 from the surface of the brain toward the tumor 31. FIG. 15 is a diagram illustrating a simulation image on the path 60 on the way from the surface of the brain to the tumor 31. As illustrated in FIG. 15, in a simulation image 58, the tissues inside the brain are shown in a region 58A surrounded by a circle. The surface of the brain is revealed outside the region 58A. As illustrated in FIG. 15, blood vessels 61, nerves 62, and so on in the brain are visually recognizable in the region 58A. In the simulation image 58 illustrated in FIG. 15, since the point of view has not reached the tumor 31, the tumor 31 is translucent (i.e., the broken line). The path 60 is shorter than that in the simulation image 57 illustrated in FIG. 14.

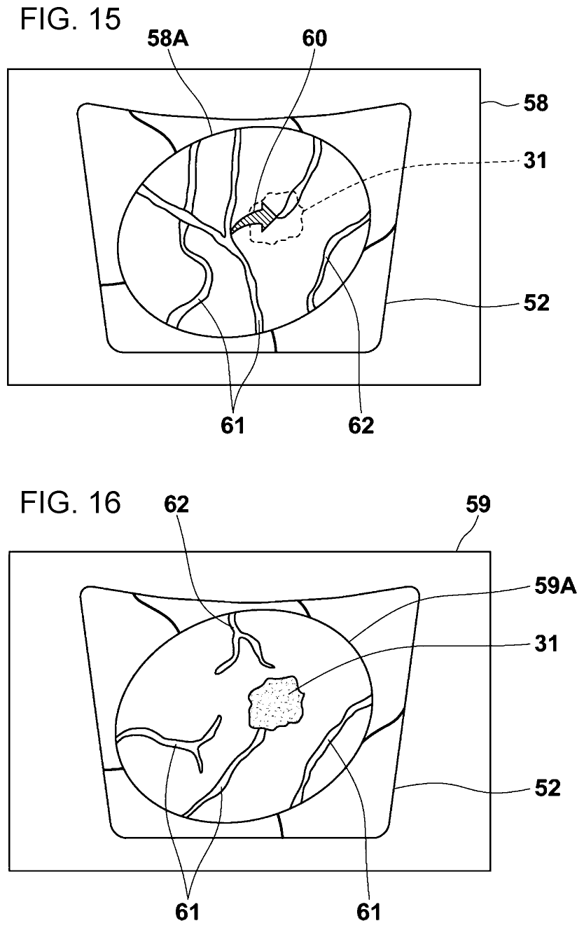

FIG. 16 illustrates a simulation image in which the point of view is shifted toward the tumor 31 by the operator further operating the input unit 15. As illustrated in FIG. 16, in a simulation image 59, the tissues inside the brain are shown in a region 59A surrounded by a circle. As in FIG. 15, the surface of the brain is revealed outside the region 59A. As illustrated in FIG. 16, the blood vessels 61, the nerves 62, and so on in the brain are visually recognizable in the region 59A. In addition, the tumor 31 is visually recognizable (i.e., the solid line).

In the foregoing description, a path from the start position C2 on the surface of the brain to the tumor 31 is sequentially displayed as simulation images. However, a simulation can be performed in the reverse direction from the state illustrated in FIG. 16 in which the tumor 31 is visible to the state illustrated in FIG. 11.

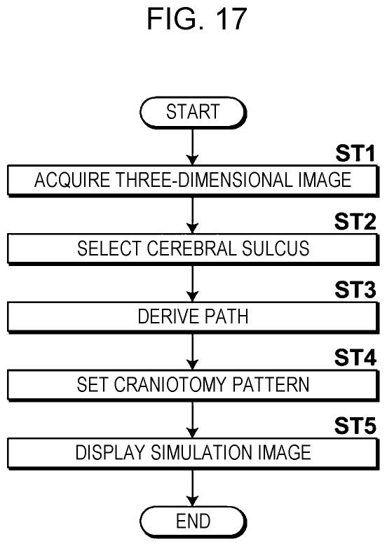

Next, a process performed in this embodiment will be described. FIG. 17 is a flowchart illustrating a process performed in this embodiment. First, the image acquisition unit 21 acquires the three-dimensional image G0 (step ST1), and the path derivation unit 22 selects at least one cerebral sulcus within a predetermined range from the center-of-gravity position C0 of the tumor 31 included in the three-dimensional image G0 (step ST2). Then, the path derivation unit 22 derives the path P0 from the tumor 31 to the surface of the brain through the selected cerebral sulcus 36A (step ST3).

Further, the craniotomy pattern setting unit 23 sets a craniotomy pattern for tracing the path P0, on the surface of the head of the patient included in the three-dimensional image G0 (step ST4). Then, the display control unit 24 displays a simulation image on the display unit 14 (step ST5), and the process ends.

In this embodiment, as described above, in the three-dimensional image G0 of the brain of a patient including an abnormal area, at least one path P0 from the abnormal area, such as the tumor 31, to the surface of the brain through a cerebral sulcus in the brain is derived, and a craniotomy pattern for tracing the path P0 is set on the surface of the head of the patient included in the three-dimensional image G0. It is therefore possible to simulate a path from the craniotomy position to the abnormal area without repeating a simulation such as changing the craniotomy position. According to this embodiment, therefore, a path to an abnormal area can be efficiently determined for a simulation of a craniotomy.

In this embodiment, furthermore, a simulation image in which the point of view is shifted from the surface of the head of the subject to the abnormal area along the path is displayed. Therefore, a path to a tumor when a surgery is performed can be checked before the surgery.

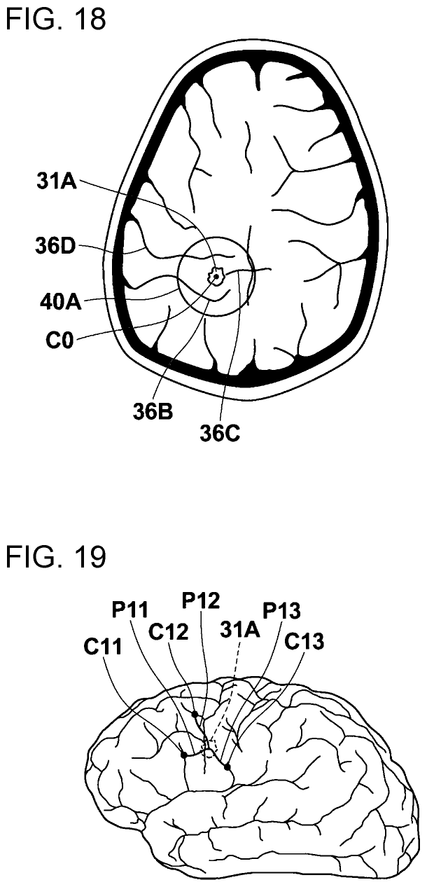

In the embodiment described above, the path derivation unit 22 derives one path P0. However, this is not limiting, and a plurality of paths may be derived. For example, as illustrated in FIG. 18, in a case where a plurality of cerebral sulci are included in a predetermined range indicated by a sphere 40A centered on the center-of-gravity position C0 of a tumor 31A, all the cerebral sulci may be selected, and a path may be derived for each of the selected cerebral sulci. In FIG. 18, three cerebral sulci 36B to 36D are selected. In this case, the path derivation unit 22 derives a path for each of the selected cerebral sulci 36B to 36D. FIG. 19 is a diagram illustrating respective paths derived for the three cerebral sulci 36B to 36D. As illustrated in FIG. 19, the path derivation unit 22 derives a path P11 from the tumor 31A to a start position C11 through the cerebral sulcus 36B, a path P12 from the tumor 31A to a start position C12 through the cerebral sulcus 36C, and a path P13 from the tumor 31A to a start position C13 through the cerebral sulcus 36D.

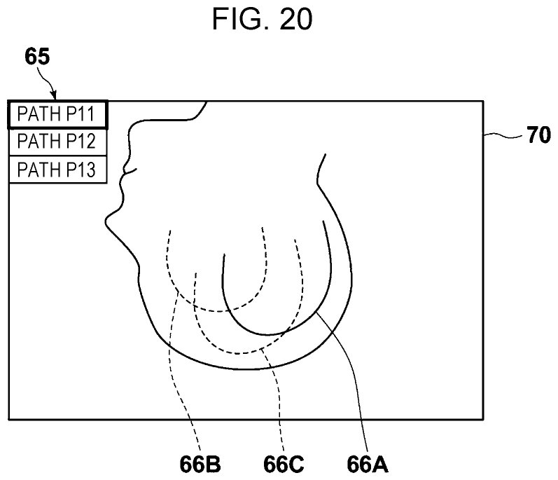

The paths P11 to P13 have different distances from the tumor 31A to the cerebral sulci 36B to 36D, respectively. Thus, the display control unit 24 sorts the plurality of paths P11 to P13 in accordance with the distances from the tumor 31A to the cerebral sulci 36B to 36D and displays a sorting result on the display unit 14 in ascending order of distance. FIG. 20 is a diagram illustrating a simulation image in which a sorting result is displayed. As illustrated in FIG. 20, a sorting result 65 is displayed in a simulation image 70. In the sorting result 65, the paths P11, P12, and P13 are arranged in this order from top to bottom in ascending order of the distance the tumor 31A to the cerebral sulcus. When the operator selects a desired path in the sorting result 65, a craniotomy pattern corresponding to the path is displayed in the simulation image 70. FIG. 20 illustrates a state in which the uppermost path P11 is selected, and a craniotomy pattern 66A corresponding to the path P11 is displayed as a solid line in the simulation image 70. Craniotomy patterns 66B and 66C for the paths P12 and P13 other than the path P11 are indicated by broken lines in the simulation image 70. When the operator selects, in the sorting result 65, a path to be displayed, a craniotomy pattern corresponding to the selected path is displayed as a solid line.

In the foregoing description, the paths P11 to P13 are sorted in ascending order of the distance from the tumor 31A to the cerebral sulcus, but this is not limiting. The paths P11 to P13 may be sorted in descending order of the distance from the tumor 31A to the cerebral sulcus. Alternatively, the paths P11 to P13 may be sorted in ascending order of the distance from the tumor 31A to each of the positions C11 to C13 on the surface of the brain, or the paths P11 to P13 may be sorted in descending order of the distance from the tumor 31A to each of the positions C11 to C13 on the surface of the brain.

In the embodiment described above, furthermore, a template is selected from a plurality of templates in accordance with the position of a derived path on the surface of the brain, and a craniotomy pattern is set on the basis of the selected template, but this is not limiting. A craniotomy pattern may be set in accordance with the position of a derived path on the surface of the brain without using templates.

In the embodiment described above, furthermore, depending on the preference of the surgical operator, there may be a cerebral sulcus that the surgical operator wishes to use before reaching a tumor. In such a case, a path that does not use a designated cerebral sulcus may be derived using settings from the input unit 15. For example, in a case where, as illustrated in FIG. 18, the three cerebral sulci 36B to 36D are selected on the basis of the tumor 31A, if the surgical operator performs setting such that the surgical operator does not wish to use the specific cerebral sulcus 36D, the path derivation unit 22 derives the paths P11 and P12, which pass through only the cerebral sulcus 36B and the cerebral sulcus 36C. This makes it possible to derive a path according to the preference of the surgical operator.

In the embodiment described above, furthermore, organs such as nerves and cerebral arteries may be present in a cerebral sulcus. A cerebral sulcus in which such organs are present is not preferably used for a craniotomy. Accordingly, the path derivation unit 22 may determine, for a selected cerebral sulcus, whether organs such as nerves and cerebral arteries are present in the cerebral sulcus, and derive a path that avoids the organs when the organs are present. In this case, a path passing through a cerebral sulcus other than the cerebral sulcus in which such organs are present is derived.

In the embodiment described above, furthermore, the three-dimensional image G0 in which an abnormal area has been detected, is saved in the image storage server 3, but this is not limiting. The craniotomy simulation device according to this embodiment may be provided with a CAD for detecting an abnormal area, and the craniotomy simulation device according to this embodiment may detect an abnormal area.

In the embodiments described above, for example, the hardware structure of processing units that execute various kinds of processing, such as the image acquisition unit 21, the path derivation unit 22, the craniotomy pattern setting unit 23, and the display control unit 24 can be implemented using the following various processors. As described above, the various processors described above include, in addition to a CPU, which is a general-purpose processor configured to execute software (program) to function as various processing units, a programmable logic device (PLD) such as an FPGA (Field Programmable Gate Array), which is a processor whose circuit configuration is changeable after manufacture, a dedicated electric circuit, which is a processor having a circuit configuration specifically designed to execute specific processing, such as an ASIC (Application Specific Integrated Circuit), and so on.

A single processing unit may be configured by one of the various processors or may be configured by a combination of two or more processors of the same type or different types (for example, a combination of a plurality of FPGAs or a combination of a CPU and an FPGA). Alternatively, a plurality of processing units may be configured by a single processor.

Examples of configuring a plurality of processing units by a single processor include, first, a form in which, as typified by a computer such as a client and a server, the single processor is configured by a combination of one or more CPUs and software and the processor functions as a plurality of processing units. The examples include, second, a form in which, as typified by a system on chip (SoC) or the like, a processor is used in which the functions of the entire system including the plurality of processing units are implemented by a single IC (Integrated Circuit) chip. As described above, the various processing units are configured using one or more of the various processors described above as a hardware structure.

The hardware structure of these various processors can be implemented by, more specifically, an electric circuit (circuitry) made by a combination of circuit elements such as semiconductor elements.

### REFERENCE SIGNS LIST

1 craniotomy simulation device

2 three-dimensional imaging device

3 image storage server

4 network

11 CPU

12 memory

13 storage

14 display

15 input unit

21 image acquisition unit

22 path derivation unit

23 craniotomy pattern setting unit

24 display control unit

30 brain

31, 31A tumor

32 tomographic image

33 skull

34 brain parenchyma

35 cerebrospinal fluid

36 cerebral sulcus

36A to 36D selected cerebral sulcus

40, 40A sphere

41 bottom of cerebral sulcus

49, 55 to 59, 70 simulation image

50 craniotomy pattern

51 incision line of skull in template

52 incision line of skull

53 incision line of skin in template

54 incision line of skin

58A, 59A region

60 path

61 blood vessel

62 nerve

65 sorting result

66A, 66B, 66C craniotomy pattern

C0 center-of-gravity position

C1 position

C2, C11 to C13 start position

G0 three-dimensional image

O origin

P0 path

P1, P2 shortest distance

P11, P12, P13 path

T1 to T5 template

What is claimed is:

1. A craniotomy simulation device comprising:
a processor configured to
derive, in a three-dimensional image of a brain of a subject including an abnormal area, at least one path from the abnormal area to a surface of the brain through a cerebral sulcus in the brain; and
set a craniotomy pattern for tracing the path, on a surface of a head of the subject included in the three-dimensional image.

2. The craniotomy simulation device according to claim 1,
wherein the processor is configured to set the craniotomy pattern on the basis of a template selected from templates representing a plurality of standard craniotomy patterns respectively in accordance with a position of the path on the surface of the brain.

3. The craniotomy simulation device according to claim 2,
wherein the processor is configured to correct the selected template in accordance with a shape of the head of the subject to set the craniotomy pattern.

4. The craniotomy simulation device according to claim 1,
wherein the processor is configured to select at least one cerebral sulcus within a predetermined range from a position of the abnormal area and derives the path that passes through the selected cerebral sulcus.

5. The craniotomy simulation device according to claim 1,
wherein the processor is configured to derive the path that passes through a cerebral sulcus other than a cerebral sulcus selected.

6. The craniotomy simulation device according to claim 1,
wherein the processor is configured to derive the path that avoids an organ designated.

7. The craniotomy simulation device according to claim 1, the processor is further configured to
display a three-dimensional image of the head of the subject for which the craniotomy pattern is set, on a display unit as a simulation image.

8. The craniotomy simulation device according to claim 7,
wherein the processor is configured to display, on the display unit, the simulation image in which a point of view is shifted from the surface of the head of the subject to the abnormal area along the path.

9. The craniotomy simulation device according to claim 7,
wherein the processor is configured to display, on the display unit, the simulation image in which the path is highlighted.

10. The craniotomy simulation device according to claim 1,
wherein the processor is configured to:
derive a plurality of the paths;
set the craniotomy pattern for each of the plurality of paths; and
sort the plurality of paths in accordance with a distance from the abnormal area to a cerebral sulcus and display a sorting result on a display unit.

11. The craniotomy simulation device according to claim 1,
wherein the processor is configured to:
derive a plurality of the paths;
set the craniotomy pattern for each of the plurality of paths; and
sort the plurality of paths in accordance with a distance from the abnormal area to the surface of the brain and display a sorting result on a display unit.

12. A craniotomy simulation method comprising:
deriving, in a three-dimensional image of a brain of a subject including an abnormal area, at least one path from the abnormal area to a surface of the brain through a cerebral sulcus in the brain; and
setting a craniotomy pattern for tracing the path, on a surface of a head of the subject included in the three-dimensional image.

13. A non-transitory computer readable recording medium storing a craniotomy simulation program for causing a computer to execute
a procedure for deriving, in a three-dimensional image of a brain of a subject including an abnormal area, at least one path from the abnormal area to a surface of the brain through a cerebral sulcus in the brain; and
a procedure for setting a craniotomy pattern for tracing the path, on a surface of a head of the subject included in the three-dimensional image.
